(19) United States
(12) Patent Application Publication
(10) Pub. No.: US 2006/0184199 A1
(43) Pub. Date: Aug. 17, 2006

(54) Apparatus and methods for reducing bleeding from a cannulation site

(76) Inventors: Shawn O'Leary, Mission Viejo, CA (US); Wolfgang Werner, Carlsbad, CA (US); Anthony Viole, Foothill Ranch, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/057,692

(22) Filed: Feb. 14, 2005

(51) Int. Cl. A61B 17/08

(52) U.S. Cl. 606/213

(57) ABSTRACT

To reduce the risk of hematoma formation, apparatus and methods for reducing bleeding from a cannulation site are provided. In one embodiment, the apparatus comprises a plug that applies pressure to the cannulation site. In another embodiment, the apparatus further comprises a hemostatic agent. In another embodiment, the hemostatic agent is flowable through a lumen of the plug to be deployed at the cannulation site.

### BACKGROUND OF THE INVENTION

1. Field of the Invention

The present invention relates to cannulation and means for minimizing bleeding that may occur during cannulation.

2. Description of the Related Art

At a vasculature cannulation site, some bleeding may occur from the vessel that the cannula penetrates. Additional bleeding may occur from the tissue that lies between the patient's skin and the vessel. Either may cause hematoma formation, which is undesirable. Prior efforts to reduce such undesirable effects have involved pressure bandages or sandbags placed over the area to minimize or stop bleeding. These and other prior methods tend to impair blood flow to the vasculature downstream of the penetration site.

### SUMMARY OF THE INVENTION

There is a need for apparatuses and methods of reducing bleeding during cannulation to reduce hematoma formation without impairing blood flow to the vasculature downstream of the penetration site.

The preferred embodiments of the present apparatus and methods for reducing bleeding from a cannulation site have several features, no single one of which is solely responsible for their desirable attributes. Without limiting the scope of these apparatus and methods as expressed by the claims that follow, their more prominent features will now be discussed briefly. After considering this discussion, and particularly after reading the section entitled “Detailed Description of the Preferred Embodiments,” one will understand how the features of the preferred embodiments provide advantages, which include reduced bleeding from a cannulation site, thereby providing a reduced likelihood of hematoma formation.

An embodiment of the present apparatus for reducing or preventing bleeding from a percutaneous cannula insertion site comprises an assembly comprising a cannula and a plug. The cannula has a proximal end and a distal end and is configured to extend into a blood vessel and provide fluid a path to an interior of the vessel. The plug is disposed about and at least partially overlapping a portion of the cannula. The plug has a distal end configured to abut an exterior wall of the vessel while the cannula is maintained within the vessel. The plug thereby reduces or prevents bleeding from the vessel. Another embodiment of the present apparatus employs a plug configured to accept a flow of hemostatic agent therethrough. The plug is disposed about and at least partially overlaps a portion (e.g., a proximal portion) of the cannula. An exterior wall of the cannula and an interior wall of the plug define an annular space.

The present invention also comprises a method for reducing or eliminating bleeding from a percutaneous cannula insertion site, wherein one such method comprises the step of inserting a cannula into a vessel such that the cannula provides a fluid path through a vessel opening to an interior of the vessel, whereby the cannula has a plug disposed about a portion (e.g., a proximal portion) thereof. The method further comprises the step of advancing the cannula into the vessel until a distal end of the plug abuts an outer wall of the vessel surrounding the vessel opening. An alternative method comprises using a cannula having a plug disposed about a proximal portion thereof, whereby an outer surface of the cannula and an inner surface of the plug define boundaries of an annular space. The method further comprises the steps of advancing the cannula into the vessel until a distal end of the plug abuts an outer wall of the vessel surrounding the vessel opening, and injecting a hemostatic agent into the annular space.

### BRIEF DESCRIPTION OF THE DRAWINGS

The preferred embodiments of the present apparatus and methods for reducing bleeding from a cannulation site, illustrating their features, will now be discussed in detail. These embodiments depict the novel and non-obvious apparatus and methods shown in the accompanying drawings, which are for illustrative purposes only. These drawings include the following figures, in which like numerals indicate like parts:

FIG. 1 is a schematic view of one embodiment of a heart assist system having multiple conduits for multi-site application, shown applied to a patient's vascular system;

FIG. 2 is a schematic view of another application of the embodiment of FIG. 1;

FIG. 3 is a schematic view of another embodiment of a heart assist system having multiple conduits for multi-site application wherein each of the conduits is applied to more than one vessel, shown applied to a patient's vascular system;

FIG. 4 is a schematic view of another embodiment of a heart assist system having multiple conduits for multi-site application and employing a connector with a T-shaped fitting, shown applied to a patient's vascular system;

FIG. 5 is a schematic view of an L-shaped connector coupled with an inflow conduit, shown inserted within a blood vessel;

FIG. 6 is a schematic view of another embodiment of a heart assist system having multiple conduits for multi-site application, shown applied to a patient's vascular system;

FIG. 7 is a schematic view of another application of the embodiment of FIG. 6, shown applied to a patient's vascular system;

FIG. 8 is a schematic view of another application of the embodiment of FIG. 6, shown applied to a patient's vascular system;

FIG. 9 is a schematic view of another embodiment of a heart assist system having multiple conduits for multi-site application, a reservoir, and a portable housing for carrying a portion of the system directly on the patient;

FIG. 10 is a schematic view of another embodiment of a heart assist system having a multilumen cannula for single-site application, shown applied to a patient's vascular system;

FIG. 11 is a schematic view of a modified embodiment of the heart assist system of FIG. 10, shown applied to a patient's vascular system;

FIG. 12 is a schematic view of another embodiment of a heart assist system having multiple conduits for single-site application, shown applied to a patient's circulatory system;

FIG. 13 is a schematic view of another application of the embodiment of FIG. 12, shown applied to a patient's vascular system;

FIG. 14 is a schematic view of one application of an embodiment of a heart assist system having an intravascular pump enclosed in a protective housing, wherein the intravascular pump is inserted into the patient's vasculature through a non-primary vessel;

FIG. 15 is a schematic view of another embodiment of a heart assist system having an intravascular pump housed within a conduit having an inlet and an outlet, wherein the intravascular pump is inserted into the patient's vasculature through a non-primary vessel;

FIG. 16 is a schematic view of a modified embodiment of the heart assist system of FIG. 15 in which an additional conduit is shown adjacent the conduit housing the pump, and in which the pump comprises a shaft-mounted helical thread;

FIG. 17 is a schematic side elevational view of an embodiment of an apparatus for reducing bleeding from a cannulation site, illustrating the apparatus deployed within a cannulation site;

FIG. 18 is a schematic side elevational view of another embodiment of an apparatus for reducing bleeding from a cannulation site, illustrating the apparatus deployed within a cannulation site;

FIG. 19 is a cross-sectional schematic view of the apparatus of FIG. 18, taken along the line 19-19;

FIG. 20 is a cross-sectional schematic view of one variation of the apparatus of FIG. 18;

FIG. 21 is a cross-sectional schematic view of another variation of the apparatus of FIG. 18;

FIG. 22 is a cross-sectional schematic view of another variation of the apparatus of FIG. 18; and

FIG. 23 is a schematic side elevation view of another embodiment of an apparatus for reducing bleeding from a cannulation site, illustrating the apparatus deployed within a cannulation site.

### DETAILED DESCRIPTION OF THE PREFERRED EMBODIMENTS

With reference to the drawings provided herein, detailed descriptions of various embodiments of heart assist systems and cannulae for use therewith are provided below. In addition, detailed descriptions of various embodiments of apparatus and methods for reducing bleeding from a cannulation site are also provided below.

### I. Extracardiac Heart Assist Systems and Methods

Below, a variety of cannulae and cannula assemblies are described that can be used in connection with a variety of heart assist systems that supplement perfusion. Such systems preferably are extracardiac in nature. In other words, the systems supplement blood perfusion, without the need to interface directly with the heart and aorta. Thus, the systems can be applied without major invasive surgery. The systems also lessen the hemodynamic burden or workload on the heart by reducing afterload, impedence, and/or left ventricular end diastolic pressure and volume (preload). The systems also advantageously increase peripheral organ perfusion and provide improvement in neurohormonal status. As discussed more fully below, the systems can be applied using one or more cannulae, one or more vascular grafts, and a combination of one or more cannulae and one or more vascular grafts. For systems employing cannula(e), the cannula(e) can be applied through multiple percutaneous insertion sites (sometimes referred to herein as a multi-site application) or through a single percutaneous insertion site (sometimes referred to herein as a single-site application).

A. Heart Assist Systems and Methods Employing Multi-site Application

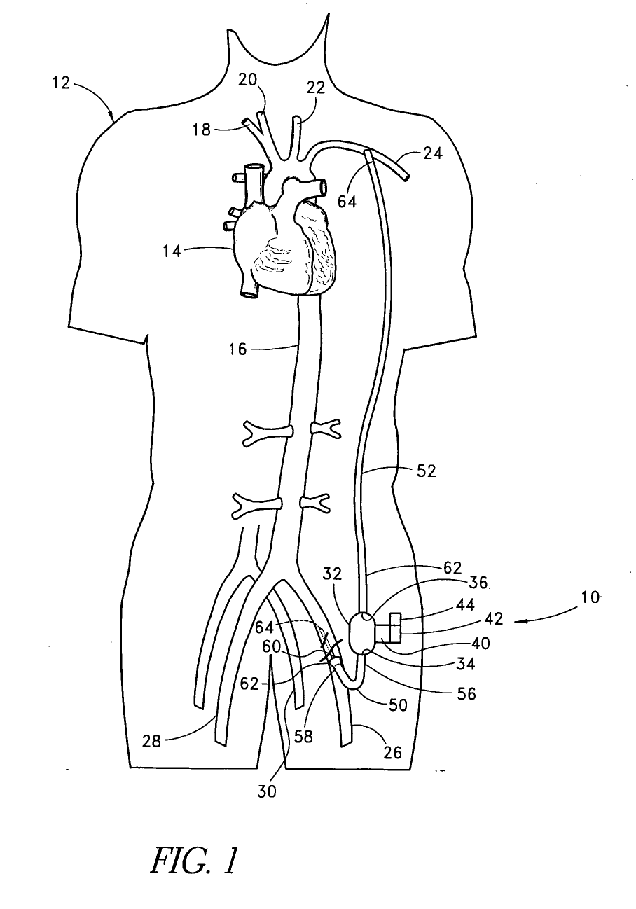

With reference to FIG. 1, a first embodiment of a heart assist system 10 is shown applied to a patient 12 having an ailing heart 14 and an aorta 16, from which peripheral brachiocephalic blood vessels extend, including the right subclavian artery 18, the right carotid artery 20, the left carotid artery 22, and the left subclavian artery 24. Extending from the descending aorta is another set of peripheral blood vessels, the left and right iliac arteries which transition into the left and right femoral arteries 26, 28, respectively. Each of the arteries 16, 18, 20, 22, 24, 26, and 28 generally conveys blood away from the heart. The vasculature includes a venous system that generally conveys blood to the heart. As discussed in more detail below, the heart assist systems described herein can also be applied to non-primary veins, including the left femoral vein 30.

The heart assist system 10 comprises a pump 32, having an inlet 34 and an outlet 36 for connection of conduits thereto. The pump 32 preferably is a rotary pump, either an axial type or a centrifugal type, although other types of pumps may be used, whether commercially-available or customized. The pump 32 preferably is sufficiently small to be implanted subcutaneously and preferably extrathoracically, for example in the groin area of the patient 12, without the need for major invasive surgery. Because the heart assist system 10 is an extracardiac system, no valves are necessary. Any inadvertent backflow through the pump 32 and/or through the inflow conduit would not harm the patient 12.

Regardless of the style or nature chosen, the pump 32 is sized to generate blood flow at subcardiac volumetric rates, less than about 50% of the flow rate of an average healthy heart, although flow rates above that may be effective. Thus, the pump 32 is sized and configured to discharge blood at volumetric flow rates anywhere in the range of 0.1 to 3 liters per minute, depending upon the application desired and/or the degree of need for heart assist. For example, for a patient experiencing advanced congestive heart failure, it may be preferable to employ a pump that has an average subcardiac rate of 2.5 to 3 liters per minute. In other patients, particularly those with minimal levels of heart failure, it may be preferable to employ a pump that has an average subcardiac rate of 0.5 liters per minute or less. In yet other patients it may be preferable to employ a pump that is a pressure wave generator that uses pressure to augment the flow of blood generated by the heart.

In one embodiment, the pump 32 is a continuous flow pump that superimposes continuous blood-flow on the pulsatile aortic blood-flow. In another embodiment, the pump 32 has the capability of synchronous actuation; i.e., it may be actuated in a pulsatile mode, either in copulsating or counterpulsating fashion.

For copulsating action, it is contemplated that the pump 32 would be actuated to discharge blood generally during systole, beginning actuation, for example, during isovolumic contraction before the aortic valve opens or as the aortic valve opens. The pump 32 would be static while the aortic valve is closed following systole, ceasing actuation, for example, when the aortic valve closes.

For counterpulsating actuation, it is contemplated that the pump 32 would be actuated generally during diastole, ceasing actuation, for example, before or during isovolumic contraction. Such an application would permit and/or enhance coronary blood perfusion. In this application, it is contemplated that the pump 32 would be static during the balance of systole after the aortic valve is opened, to lessen the burden against which the heart must pump. The aortic valve being open encompasses the periods of opening and closing, wherein blood is flowing therethrough.

It should be recognized that the designations copulsating and counterpulsating are general identifiers and are not limited to specific points in the patient's heart cycle when the pump 32 begins and discontinues actuation. Rather, they are intended to generally refer to pump actuation in which the pump 32 is actuating, at least in part, during systole and diastole, respectively. For example, it is contemplated that the pump 32 might be activated to be out of phase from true copulsating or counterpulsating actuation described herein, and still be synchronous, depending upon the specific needs of the patient or the desired outcome. One might shift actuation of the pump 32 to begin prior to or after isovolumic contraction or to begin before or after isovolumic relaxation.

Furthermore, the pulsatile pump may be actuated to pulsate asynchronously with the patient's heart. Typically, where the patient's heart is beating irregularly, there may be a desire to pulsate the pump 32 asynchronously so that the perfusion of blood by the heart assist system 10 is more regular and, thus, more effective at oxygenating the organs. Where the patient's heart beats regularly, but weakly, synchronous pulsation of the pump 32 may be preferred.

The pump 32 is driven by a motor 40 and/or other type of drive means and is controlled preferably by a programmable controller 42 that is capable of actuating the pump 32 in pulsatile fashion, where desired, and also of controlling the speed or output of the pump 32. For synchronous control, the patient's heart would preferably be monitored with an EKG in which feedback would be provided to the controller 42. The controller 42 is preferably programmed through external means, such as, for example, RF telemetry circuits of the type commonly used within implantable pacemakers and defibrillators. The controller may also be autoregulating to permit automatic regulation of the speed, and/or regulation of the synchronous or asynchronous pulsation of the pump 32, based upon feedback from ambient sensors monitoring parameters, such as pressure or the patient's EKG. It is also contemplated that a reverse-direction pump be used, if desired, in which the controller is capable of reversing the direction of either the drive means or the impellers of the pump. Such a pump might be used where it is desirable to have the option of reversing the direction of circulation between two blood vessels.

Power to the motor 40 and the controller 42 may be provided by a power source 44, such as a battery, that is preferably rechargeable by an external induction source (not shown), such as an RF induction coil that may be electromagnetically coupled to the battery to induce a charge therein. Alternative power sources are also possible, including a device that draws energy directly from the patient's body; e.g., the patient's muscles, chemicals or heat. The pump can be temporarily stopped during recharging with no appreciable life threatening effect, because the system only supplements the heart, rather than substituting for the heart.

While the controller 42 and power source 44 are preferably pre-assembled to the pump 32 and implanted therewith, it is also contemplated that the pump 32 and motor 40 be implanted at one location and the controller 42 and the power source 44 be implanted in a separate location. In one alternative arrangement, the pump 32 may be driven externally through a percutaneous drive line or cable, as shown in FIG. 16. In another variation, the pump, motor and controller may be implanted and powered by an extracorporeal power source. In the latter case, the power source could be attached to the side of the patient to permit fully ambulatory movement.

The inlet 34 of the pump 32 is preferably connected to an inflow conduit 50 and an outflow conduit 52 to direct blood flow from one peripheral blood vessel to another. The conduits 50, 52 preferably are flexible conduits, as discussed more fully below. The conduits 50, 52 are coupled with the peripheral vessels in different ways in various embodiments of the heart assist system 10. As discussed more fully below, at least one of the conduits 50, 52 can be connected to a peripheral vessel, e.g., as a graft, using an anastomosis connection, and at least one of the conduits 50, 52 can be coupled with the same or another vessel via insertion of a cannula into the vasculature. Also, more than two conduits are used in some embodiments, as discussed below.

The inflow and outflow conduits 50, 52 may be formed from Dacron, Hemashield, Gortex, PVC, polyurethane, PTFE, ePTFE, nylon, or PEBAX materials, although other synthetic materials may be suitable. The inflow and outflow conduits 50, 52 may also comprise biologic materials or pseudobiological (hybrid) materials (e.g., biologic tissue supported on a synthetic scaffold). The inflow and outflow conduits 50, 52 are preferably configured to minimize kinks so blood flow is not meaningfully interrupted by normal movements of the patient or compressed easily from external forces. In some cases, the inflow and/or outflow conduits 50, 52 may come commercially already attached to the pump 32. Where it is desired to implant the pump 32 and the conduits 50, 52, it is preferable that the inner diameter of the conduits 50, 52 be less than 25 mm, although diameters slightly larger may be effective.

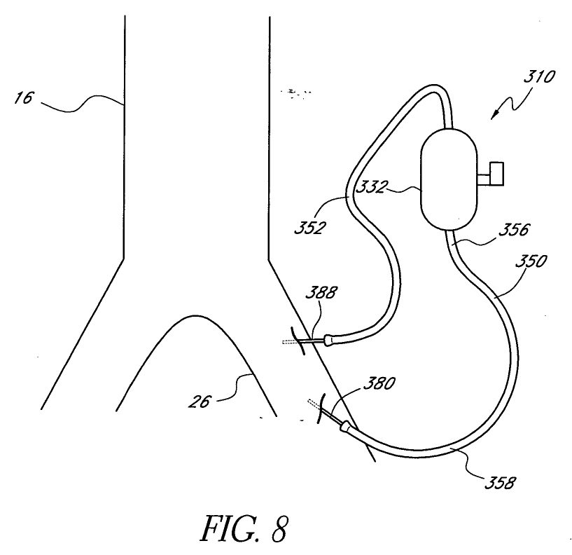

In one preferred application, the heart assist system 10 is applied in an arterial-arterial fashion; for example, as a femoral-axillary connection, as is shown in FIG. 1. Those of skill in the art will appreciate that an axillary-femoral connection would also be effective using the embodiments described herein. Indeed, those of skill in the art will appreciate that the heart assist system 10 might be applied to any of the peripheral blood vessels in the patient. Another application of the heart assist system 10 couples the conduits 50, 52 with the same non-primary vessel in a manner similar to the application shown in FIG. 8 and discussed below.

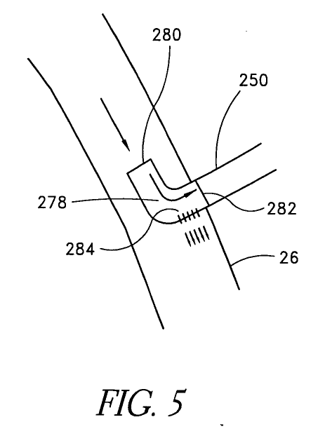

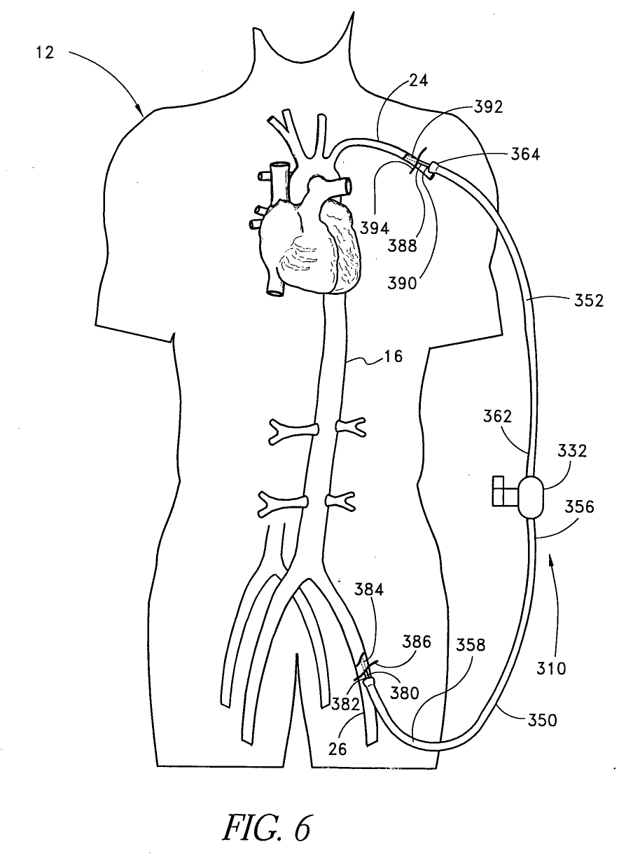

FIG. 1 shows that the inflow conduit 50 has a first end 56 that connects with the inlet 34 of the pump 32 and a second end 58 that is coupled with a first non-primary blood vessel (e.g., the left femoral artery 26) by way of an inflow cannula 60. The inflow cannula 60 has a first end 62 and a second end 64. The first end 62 is sealably connected to the second end 58 of the inflow conduit 50. The second end 64 is inserted into the blood vessel (e.g., the left femoral artery 26). Although shown as discrete structures in FIG. 1, those of skill in the art will appreciate that the inflow conduit 50 and the cannula 60 may be unitary in construction. The cannula 60 can take any suitable form, e.g., defining a lumen with an inner size that increases distally.

Where the conduit 50 is at least partially extracorporeal, the inflow cannula 60 also may be inserted through a surgical opening (e.g., as shown in FIG. 6 and described in connection therewith) or percutaneously, with or without an introducer sheath (not shown). In other applications, the inflow cannula 60 could be inserted into the right femoral artery or any other peripheral artery.

FIG. 1 shows that the outflow conduit 52 has a first end 66 that connects to the outlet 36 of the pump 32 and a second end 68 that connects with a second peripheral blood vessel, preferably the left subclavian artery 24 of the patient 12, although the right axillary artery, or any other peripheral artery, would be acceptable. In one application, the connection between the outflow conduit 52 and the second blood vessel is via an end-to-side anastomosis, although a side-to-side anastomosis connection might be used mid-stream of the conduit where the outflow conduit were connected at its second end to yet another blood vessel or at another location on the same blood vessel (neither shown). Preferably, the outflow conduit 52 is attached to the second blood vessel at an angle that results in the predominant flow of blood out of the pump 32 proximally toward the aorta 16 and the heart 14, such as is shown in FIG. 1, while still maintaining sufficient flow distally toward the hand to prevent limb ischemia.

In another embodiment, the inflow conduit 50 is connected to the first blood vessel via an end-to-side anastomosis, rather than via the inflow cannula 60. The inflow conduit 50 could also be coupled with the first blood vessel via a side-to-side anastomosis connection mid-stream of the conduit, where the inflow conduit connects at its second end to an additional blood vessel or at another location on the same blood vessel (neither shown). Further details of these arrangements and other related applications are described in U.S. application Ser. No. 10/289,467, filed Nov. 6, 2002, the entire contents of which is hereby incorporated by reference in its entirety and made a part of this specification.

In another embodiment, the outflow conduit 52 also is coupled with the second blood vessel via a cannula, as shown in FIG. 6. This connection may be achieved in a manner similar to that shown in FIG. 1 in connection with the first blood vessel.

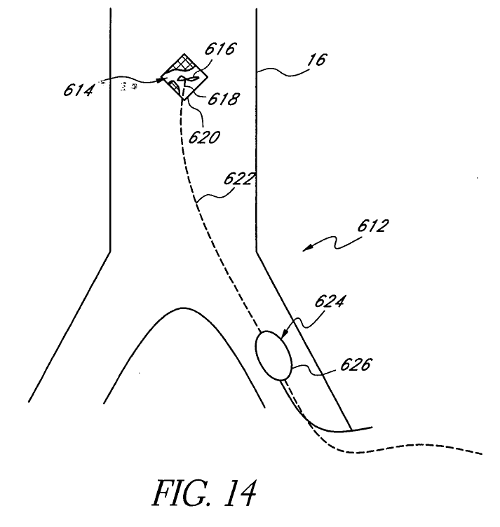

Preferably, the heart assist system 10 is applied to the peripheral or non-primary blood vessels subcutaneously; e.g., at a shallow depth just below the skin or first muscle layer so as to avoid major invasive surgery. It is also preferred that the heart assist system 10 be applied extrathoracically to avoid the need to invade the patient's chest cavity. Where desired, the entire heart assist system 10 may be implanted within the patient 12, either extravascularly, e.g., as in FIG. 1, or at least partially intravascularly, e.g., as in FIGS. 14-16.

In the case of an extravascular application, the pump 32 may be implanted, for example, into the groin area, with the inflow conduit 50 fluidly connected subcutaneously to, for example, the femoral artery 26 proximate the pump 32. The outflow conduit would be tunneled subcutaneously through to, for example, the left subclavian artery 24. In an alternative arrangement, the pump 32 and associated drive and controller could be temporarily fastened to the exterior skin of the patient, with the inflow and outflow conduits 50, 52 connected percutaneously. In either case, the patient may be ambulatory without restriction of tethered lines.

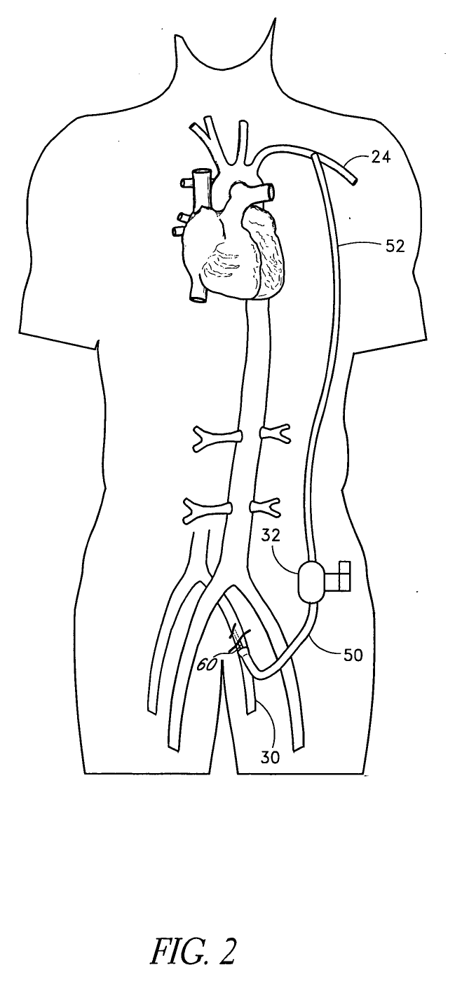

While the heart assist system 10 and other heart assist systems described herein may be applied to create an arterial-arterial flow path, given the nature of the heart assist systems, i.e., supplementation of circulation to meet organ demand, a venous-arterial flow path may also be used. For example, with reference to FIG. 2, one application of the heart assist system 10 couples the inflow conduit 50 with a non-primary vein of the patient 12, such as the left femoral vein 30. In this arrangement, the outflow conduit 50 may be fluidly coupled with one of the peripheral arteries, such as the left subclavian artery 24. Arterial-venous arrangements are contemplated as well.

In those venous-arterial cases where the inflow is connected to a vein and the outflow is connected to an artery, the pump 32 is preferably sized to permit flow sufficiently small so that oxygen-deficient blood does not rise to unacceptable levels in the arteries. The connections to the non-primary veins could be by one or more of the approaches described above for connecting to a non-primary artery. The extracardiac assist system could also be applied as a venous-venous flow path, wherein the inflow and outflow are connected to separate peripheral veins. In addition, an alternative embodiment comprises two discrete pumps and conduit arrangements, one being applied as a venous-venous flow path, and the other as an arterial-arterial flow path.

When venous blood is mixed with arterial blood, either at the inlet of the pump or at the outlet of the pump, the ratio of venous blood to arterial blood is preferably controlled to maintain an arterial saturation of a minimum of 80% at the pump inlet or outlet. Arterial saturation can be measured and/or monitored by pulse oximetry, laser doppler, colorimetry or other methods used to monitor blood oxygen saturation. The venous blood flow into the system can then be controlled by regulating the amount of blood allowed to pass through the conduit from the venous-side connection.

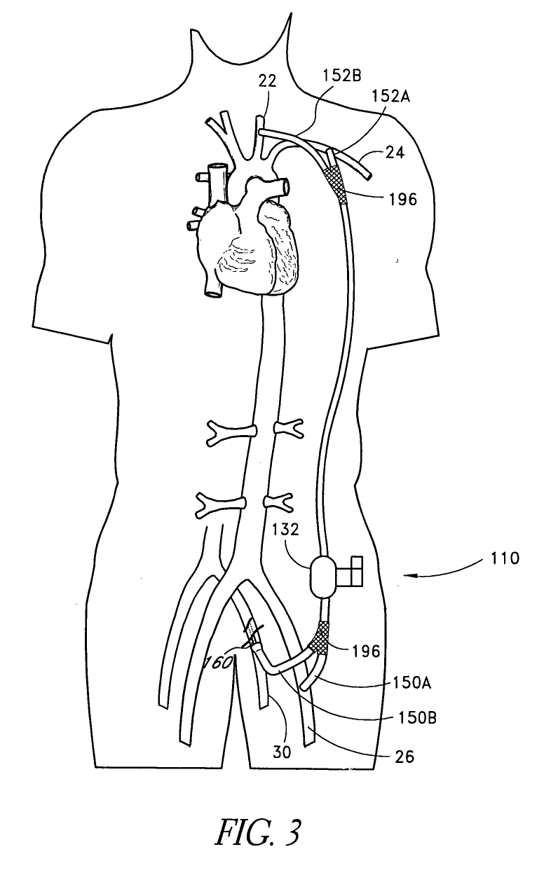

FIG. 3 shows another embodiment of a heart assist system 110 applied to the patient 12. For example, the heart assist system 110 includes a pump 132 in fluid communication with a plurality of inflow conduits 150A, 150B and a plurality of outflow conduits 152A, 152B. Each pair of conduits converges at a generally Y-shaped convergence 196 that converges the flow at the inflow end and diverges the flow at the outflow end. Each conduit may be connected to a separate peripheral blood vessel, although it is possible to have two connections to the same blood vessel at remote locations.

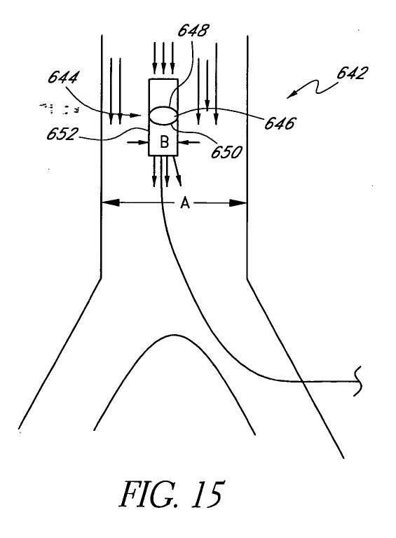

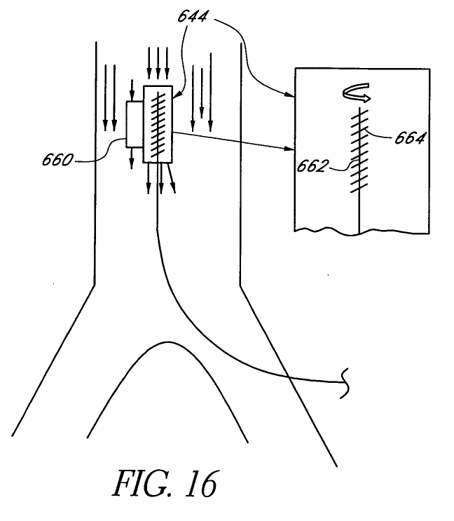

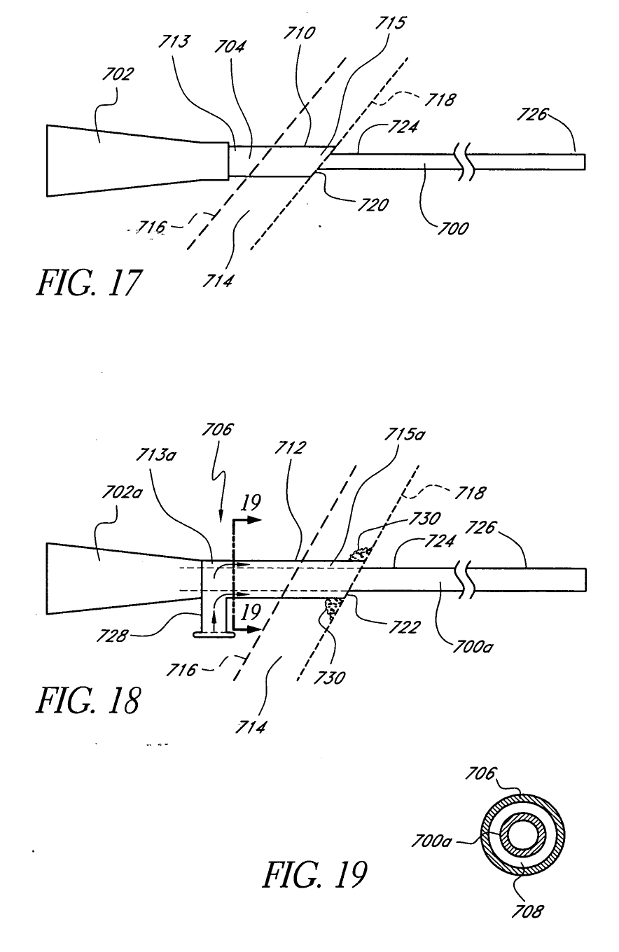

In one arrangement, all four conduits are connected to peripheral arteries. In another arrangement, one or more of the conduits could be connected to veins. In the arrangement of FIG. 3, the inflow conduit 150A is connected to the left femoral artery 26 while the inflow conduit 150B is connected to the left femoral vein 30. The outflow conduit 152A is connected to the left subclavian artery 24 while the outflow conduit 152B is connected to the left carotid artery 22. Preferably at least one of the conduits 150A, 150B, 152A, and 152B is coupled with a corresponding vessel via a cannula. In the illustrated embodiment, the inflow conduit 150B is coupled with the left femoral vein 30 via a cannula 160. The cannula 160 is coupled in a manner similar to that shown in FIG. 2 and described in connection with the cannula 60. The cannula 160 can take suitable form, e.g., defining a lumen with an inner size that increases distally as discussed below in connection with FIGS. 17-27.

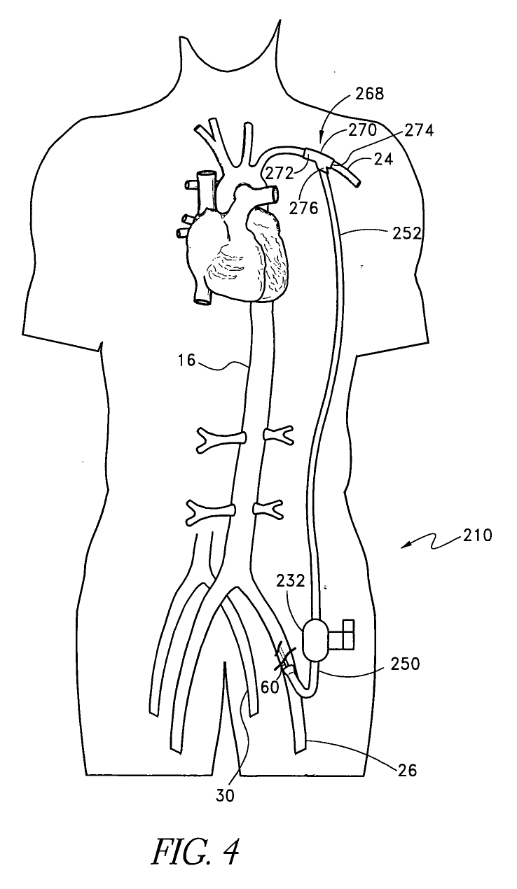

The connections of any or all of the conduits of the system 110 to the blood vessels may be via an anastomosis connection or via a connector, as described below in connection with FIG. 4. In addition, the embodiment of FIG. 3 may be applied to any combination of peripheral blood vessels that would best suit the patient's condition. For example, it may be desired to have one inflow conduit and two outflow conduits or vice versa. More than two conduits may be used on the inflow or outflow side, where the number of inflow conduits is not necessarily equal to the number of outflow conduits.

Where an anastomosis connection is not desired, a connector may be used to connect at least one of the inflow conduit and the outflow conduit to a peripheral blood vessel. With reference to FIG. 4, an embodiment of a heart assist system 210 is shown, wherein an outflow conduit 252 is connected to a non-primary blood vessel, e.g., the left subclavian artery 24, via a connector 268 that comprises a three-opening fitting. In one embodiment, the connector 268 comprises an intra-vascular, generally T-shaped fitting 270 having a proximal end 272 (relative to the flow of blood in the left axillary artery and therethrough), a distal end 274, and an angled divergence 276 permitting connection to the outflow conduit 252 and the left subclavian artery 24. The proximal and distal ends 274, 276 of the fittings 272 permit connection to the blood vessel into which the fitting is positioned, e.g., the left subclavian artery 24. The angle of divergence 276 of the fittings 272 may be 90 degrees or less in either direction from the axis of flow through the blood vessel, as optimally selected to generate the needed flow distally toward the hand to prevent limb ischemia, and to insure sufficient flow and pressure toward the aorta to provide the circulatory assistance and workload reduction needed while minimizing or avoiding endothelial damage to the blood vessel. In another embodiment, the connector 268 is a sleeve (not shown) that surrounds and attaches to the outside of the non-primary blood vessel where, within the interior of the sleeve, a port to the blood vessel is provided to permit blood flow from the outflow conduit 252 when the conduit 252 is connected to the connector 268.

Other types of connectors having other configurations are contemplated that may avoid the need for an anastomosis connection or that permit connection of the conduit(s) to the blood vessel(s). For example, it is contemplated that an L-shaped connector be used if it is desired to withdraw blood more predominantly from one direction of a peripheral vessel or to direct blood more predominantly into a peripheral vessel. Referring to FIG. 5, the inflow conduit 250 is fluidly connected to a peripheral vessel, for example, the left femoral artery 26, using an L-shaped connector 278. Of course the system 210 could be configured so that the outflow conduit 252 is coupled to a non-primary vessel via the L-shaped connector 278 and the inflow conduit 250 is coupled via a cannula, as shown in FIG. 3.

The L-shaped connector 278 has an inlet port 280 at a proximal end and an outlet port 282 through which blood flows into the inflow conduit 250. The L-shaped connector 278 also has an arrangement of holes 284 within a wall positioned at a distal end opposite the inlet port 280 so that some of the flow drawn into the L-shaped connector 278 is diverted through the holes 284, particularly downstream of the L-shaped connector 278, as in this application. A single hole 284 in the wall could also be effective, depending upon size and placement. The L-shaped connector 278 may be a deformable L-shaped catheter percutaneously applied to the blood vessel or, in an alternative embodiment, be connected directly to the walls of the blood vessel for more long term application. By directing some blood flow downstream of the L-shaped connector 278 during withdrawal of blood from the vessel, ischemic damage downstream from the connector may be avoided. Such ischemic damage might otherwise occur if the majority of the blood flowing into the L-shaped connector 278 were diverted from the blood vessel into the inflow conduit 252. It is also contemplated that a connection to the blood vessels might be made via a cannula, wherein the cannula is implanted, along with the inflow and outflow conduits.

One advantage of discrete connectors manifests in their application to patients with chronic CHF. A connector eliminates a need for an anastomosis connection between the conduits 250, 252 and the peripheral blood vessels where it is desired to remove and/or replace the system more than one time. The connectors could be applied to the first and second blood vessels semi-permanently, with an end cap applied to the divergence for later quick-connection of the cardiac assist system to the patient. In this regard, a patient might experience the benefit of the heart assist systems described herein periodically, without having to reconnect and redisconnect the conduits 250, 252 from the blood vessels via an anastomosis procedure each time. Each time it is desired to implement any of the embodiments of the heart assist system, the end caps would be removed and a conduit attached to the connector(s) quickly.

In the preferred embodiment of the connector 268, the divergence 276 is oriented at an acute angle significantly less than 90 degrees from the axis of the T-shaped fitting 270, as shown in FIG. 4, so that a majority of the blood flowing through the outflow conduit 252 into the blood vessel (e.g., left subclavian artery 24) flows in a direction proximally toward the heart 14, rather than in the distal direction. In an alternative embodiment, the proximal end 272 of the T-shaped fitting 270 may have a diameter larger than the diameter of the distal end 274, without need of having an angled divergence, to achieve the same result.

With or without a connector, with blood flow directed proximally toward the aorta 16, the result may be concurrent flow down the descending aorta, which will result in the reduction of afterload, impedence, and/or reducing left ventricular end diastolic pressure and volume (preload). Thus, the heart assist systems described herein may be applied so to reduce the afterload on the patient's heart, permitting at least partial if not complete CHF recovery, while supplementing blood circulation. Concurrent flow depends upon the phase of operation of the pulsatile pump and the choice of second blood vessel to which the outflow conduit is connected.

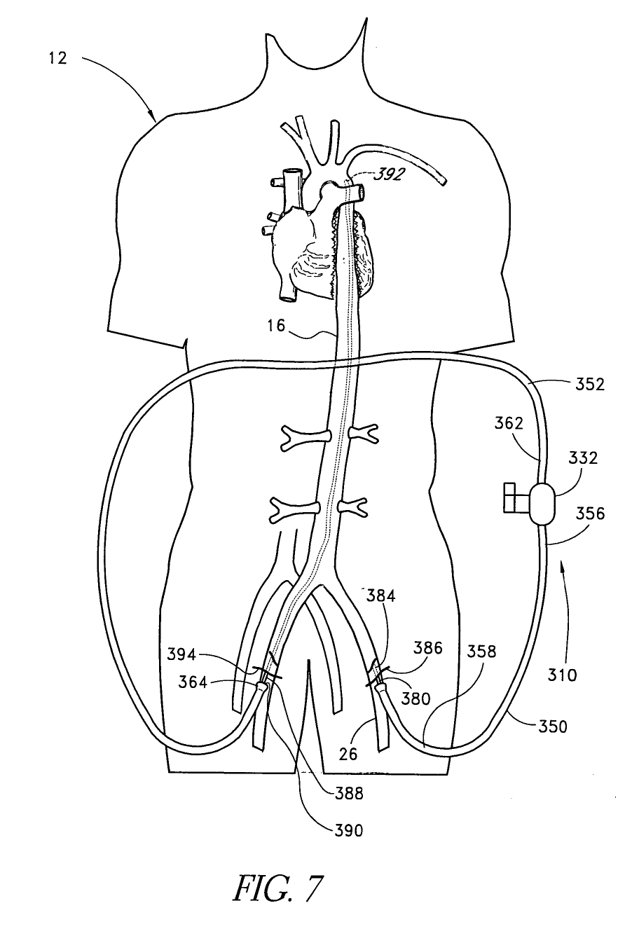

A partial external application of the heart assist systems may be appropriate where a patient with heart failure is suffering an acute decompensation episode; i.e., is not expected to survive long, or in the earlier stages of heart failure (where the patient is in New York Heart Association Classification (NYHAC) functional classes II or III). With reference to FIGS. 6 and 7, another embodiment of a heart assist system 310 is applied percutaneously to a patient 312 to connect two non-primary blood vessels wherein a pump 332 and its associated driving means and controls are employed extracorporeally.

The pump 332 has an inflow conduit 350 and an outflow conduit 352 associated therewith for connection to two non-primary blood vessels. The inflow conduit 350 has a first end 356 and a second end 358 wherein the second end 358 is connected to a first non-primary blood vessel (e.g., femoral artery 26) by way of an inflow cannula 380. The inflow cannula 380 has a first end 382 sealably connected to the second end 358 of the inflow conduit 350. The inflow cannula 380 also has a second end 384 that is inserted through a surgical opening 386 or an introducer sheath (not shown) and into the blood vessel (e.g., the left femoral artery 26).

Similarly, the outflow conduit 352 has a first end 362 and a second end 364 wherein the second end 364 is connected to a second non-primary blood vessel (e.g., the left subclavian artery 24, as shown in FIG. 6, or the right femoral artery 28, as shown in FIG. 7) by way of an outflow cannula 388. Like the inflow cannula 380, the outflow cannula 388 has a first end 390 sealably connected to the second end 364 of the outflow conduit 352. The outflow cannula 388 also has a second end 392 that is inserted through surgical opening 394 or an introducer sheath (not shown) and into the second blood vessel (e.g., the left subclavian artery 24 or the right femoral artery 28). The cannulae 380 and 388 can take any suitable form, e.g., defining a lumen with an inner size that increases distally.

As shown in FIG. 7, the second end 392 of the outflow cannula 388 may extend well into the aorta 16 of the patient 12, for example, proximal to the left subclavian artery. If desired, it may also terminate within the left subclavian artery or the left axillary artery, or in other blood vessels, such as the mesenteric or renal arteries (not shown), where in either case, the outflow cannula 388 has passed through at least a portion of a primary artery (in this case, the aorta 16). Also, if desired, blood drawn into the extracardiac system 310 described herein may originate from the descending aorta (or an artery branching therefrom) and be directed into a blood vessel that is neither the aorta nor pulmonary artery. By use of a percutaneous application, the heart assist system 310 may be applied temporarily without the need to implant any aspect thereof or to make anastomosis connections to the blood vessels.

An alternative variation of the embodiment of FIG. 6 may be used where it is desired to treat a patient periodically, but for short periods of time each occasion and without the use of special connectors. With this variation, the second ends of the inflow and outflow conduits 350, 352 may be more permanently connected to the associated blood vessels via, for example, an anastomosis connection, wherein a portion of each conduit proximate to the blood vessel connection is implanted percutaneously with a removable cap enclosing the externally-exposed first end (or an intervening end thereof) of the conduit external to the patient. When it is desired to provide a circulatory flow path to supplement blood flow, the removable cap on each exposed percutaneously-positioned conduit could be removed and the pump (or the pump with a length of inflow and/or outflow conduit attached thereto) inserted between the exposed percutaneous conduits. In this regard, a patient may experience the benefit of the cardiac assist system periodically, without having to reconnect and redisconnect the conduits from the blood vessels each time.

Specific methods of applying this alternative embodiment may further comprise coupling the inflow conduit 352 upstream of the outflow conduit 350 (as shown in FIG. 8), although the reverse arrangement is also contemplated. It is also contemplated that either the cannula 380 coupled with the inflow conduit 350 or the cannula 388 coupled with the outflow conduit 352 may extend through the non-primary blood vessel to a second blood vessel (e.g., through the left femoral artery 26 to the aorta 16 proximate the renal branch) so that blood may be directed from the non-primary blood vessel to the second blood vessel or vice versa.

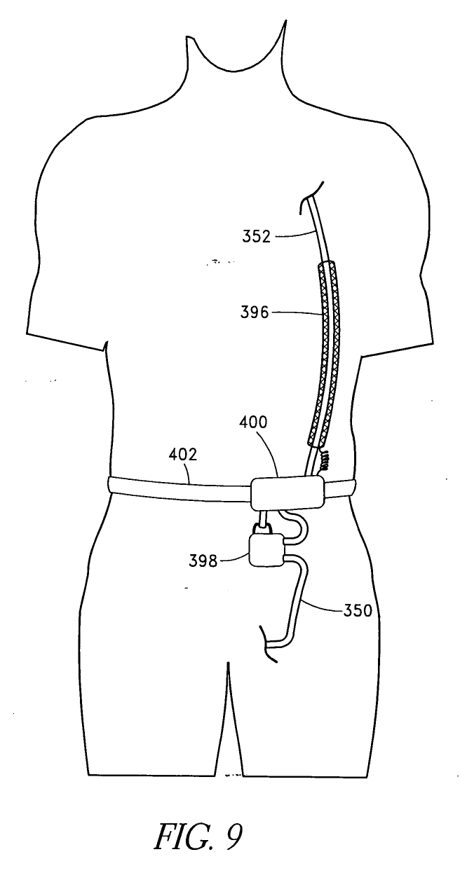

A means for minimizing the loss of thermal energy from the patient's blood may be provided where any of the heart assist systems described herein are applied extracorporeally. Such means for minimizing the loss of thermal energy may comprise, for example, a heated bath through which the inflow and outflow conduits pass or, alternatively, thermal elements secured to the exterior of the inflow and outflow conduits. Referring to FIG. 9, one embodiment comprises an insulating wrap 396 surrounding the outflow conduit 352 having one or more thermal elements passing therethrough. The elements may be powered, for example, by a battery (not shown). One advantage of thermal elements is that the patient may be ambulatory, if desired. Other means that are known by persons of ordinary skill in the art for ensuring that the temperature of the patient's blood remains at acceptable levels while traveling extracorporeally are also contemplated.

If desired, the cardiac assist system may further comprise a reservoir that is either contained within or in fluid communication with the inflow conduit. This reservoir is preferably made of materials that are nonthrombogenic. Referring to FIG. 9, a reservoir 398 is positioned fluidly in line with the inflow conduit 350. The reservoir 398 serves to sustain adequate blood in the system when the pump demand exceeds momentarily the volume of blood available in the peripheral blood vessel in which the inflow conduit resides until the pump output can be adjusted. The reservoir 398 reduces the risk of excessive drainage of blood from the peripheral blood vessel, which may occur when cardiac output falls farther than the already diminished baseline level of cardiac output, or when there is systemic vasodilation, as can occur, for example, with septic shock. It is contemplated that the reservoir 398 would be primed with an acceptable solution, such as saline, when the present system is first applied to the patient.

As explained above, one of the advantages of several embodiments of the heart assist system is that such systems permit the patient to be ambulatory. The systems may be designed to be portable, so the patient may carry the system. Referring to FIG. 9, the system may include a portable case 400 with a belt strap 402 to house the pump, power supply and/or the controller, along with certain portions of the inflow and/or outflow conduits, if necessary. Alternatively, or in addition, the system may include a shoulder strap, a backpack, a fanny pack, or other apparatus that permits effective portability. As shown in FIG. 9, blood is drawn through the inflow conduit 350 into a pump contained within the portable case 400, where it is discharged into the outflow conduit 352 back into the patient.

B. Heart Assist Systems and Methods Employing Single-site Application

As discussed above, heart assist systems can be applied to a patient through a single cannulation site. Such single-site systems can be configured with a pump located outside the vasculature of a patient, e.g., as extravascular pumping systems, inside the vasculature of the patient, e.g., as intravascular systems, or a hybrid thereof, e.g., partially inside and partially outside the vasculature of the patient.

1. Single-Site Application of Extravascular Pumping Systems

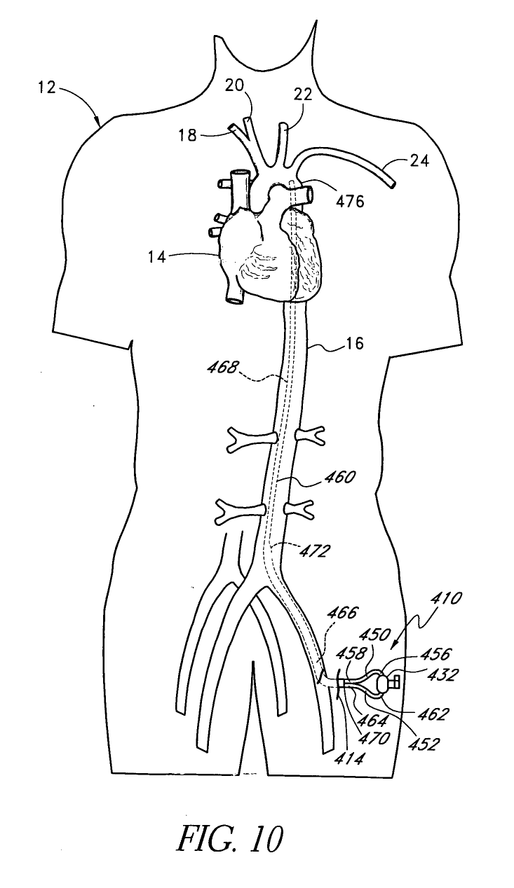

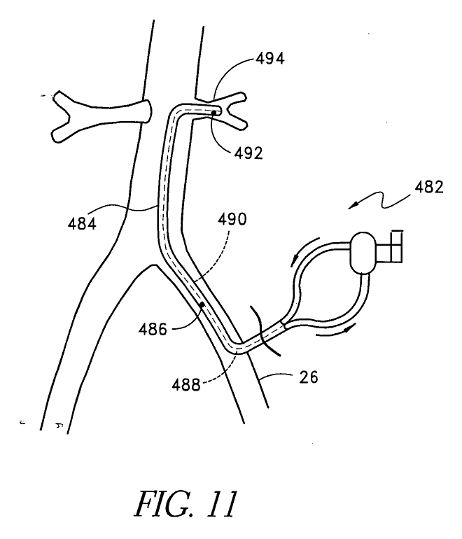

FIGS. 10 and 11 illustrate extracardiac heart assist systems that employ an extravascular pump and that can be applied as a single-site system. FIG. 10 shows a system 410 that is applied to a patient 12 through a single cannulation site 414 while inflow and outflow conduits fluidly communicate with non-primary vessels. The heart assist system 410 is applied to the patient 12 percutaneously through a single-site to couple two blood vessels with a pump 432. The pump 432 can have any of the features described above in connection with the pump 32. The pump 432 has an inflow conduit 450 and an outflow conduit 452 associated therewith. The inflow conduit 450 has a first end 456 and a second end 458. The first end 456 of the inflow conduit 450 is connected to the inlet of the pump 432 and the second end 458 of the inflow conduit 450 is fluidly coupled with a first non-primary blood vessel (e.g., the femoral artery 26) by way of a multilumen cannula 460. Similarly, the outflow conduit 452 has a first end 462 and a second end 464. The first end 462 of the outflow conduit 452 is connected to the outlet of the pump 432 and the second end 464 of the outflow conduit 452 is fluidly coupled with a second blood vessel (e.g., the descending aorta 16) by way of the multilumen cannula 460.

In one embodiment, the multilumen cannula 460 includes a first lumen 466 and a second lumen 468. The first lumen 466 extends from a proximal end 470 of the multilumen cannula 460 to a first distal end 472. The second lumen 468 extends from the proximal end 470 to a second distal end 474. In the illustrated embodiment, the second end 458 of the inflow conduit 450 is connected to the first lumen 466 of the multilumen cannula 460 and the second end 464 of the outflow conduit 452 is connected to the second lumen 468 of the multilumen cannula 460.

Where there is a desire for the patient 12 to be ambulatory, the multilumen cannula 460 preferably is made of material sufficiently flexible and resilient to permit the patient 12 to comfortably move about while the multilumen cannula 460 is indwelling in the patient's blood vessels without causing any vascular trauma.

The application shown in FIG. 10 and described above results in flow from the first distal end 472 to the second distal end 474. Of course, the flow direction may be reversed using the same arrangement, resulting in flow from the second distal end 474 to the first distal end 472. In some applications, the system 410 is applied in an arterial-arterial fashion. For example, as illustrated, the multilumen cannula 460 can be inserted into the left femoral artery 26 of the patient 12 and guided superiorly through the descending aorta to one of numerous locations. In one application, the multilumen cannula 460 can be advanced until the distal end 474 is located in the aortic arch 476 of the patient 12. The blood could discharge, for example, directly into the descending aorta proximate an arterial branch, such as the left subclavian artery or directly into the peripheral mesenteric artery (not shown).

The pump 432 draws blood from the patient's vascular system in the area near the distal end 472 and into the lumen 466. This blood is further drawn into the lumen of the conduit 450 and into the pump 432. The pump 432 then expels the blood into the lumen of the outflow conduit 452, which carries the blood into the lumen 468 of the multilumen cannula 460 and back into the patient's vascular system in the area near the distal end 474.

FIG. 11 shows another embodiment of a heart assist system 482 that is similar to the heart assist system 410, except as set forth below. The system 482 employs a multilumen cannula 484. In one application, the multilumen cannula 484 is inserted into the left femoral artery 26 and guided superiorly through the descending aorta to one of numerous locations. Preferably, the multilumen cannula 484 has an inflow port 486 that is positioned in one application within the left femoral artery 26 when the cannula 484 is fully inserted so that blood drawn from the left femoral artery 26 is directed through the inflow port 486 into a first lumen 488 in the cannula 484. The inflow port 486 can also be positioned in any other suitable location within the vasculature, described herein or apparent to one skilled in the art. This blood is then pumped through a second lumen 490 in the cannula 484 and out through an outflow port 492 at the distal end of the cannula 484.

The outflow port 492 may be situated within, for example, a mesenteric artery 494 such that blood flow results from the left femoral artery 26 to the mesenteric artery 494. The blood could discharge, for example, directly into the descending aorta proximate an arterial branch, such as the renal arteries, the left subclavian artery, or directly into the peripheral mesenteric artery 494, as illustrated in FIG. 11. Where there is a desire for the patient to be ambulatory, the multilumen cannula 484 preferably is made of material sufficiently flexible and resilient to permit the patient 12 to comfortably move about while the cannula 484 is indwelling in the patient's blood vessels without causing any vascular trauma.

Additional details of the multilumen cannula 460 may be found in U.S. patent application Ser. No. 10/078,283, filed Feb. 14, 2002, entitled A MULTILUMEN CATHETER FOR MINIMIZING LIMB ISCHEMIA, in U.S. patent application Ser. No. 10/706,346, filed Nov. 12, 2003, entitled CANNULAE HAVING REDIRECTING TIP, and in U.S. patent application Ser. No. 10/735,413, filed Dec. 12, 2003 each of which is hereby expressly incorporated by reference in its entirety and made a part of this specification.

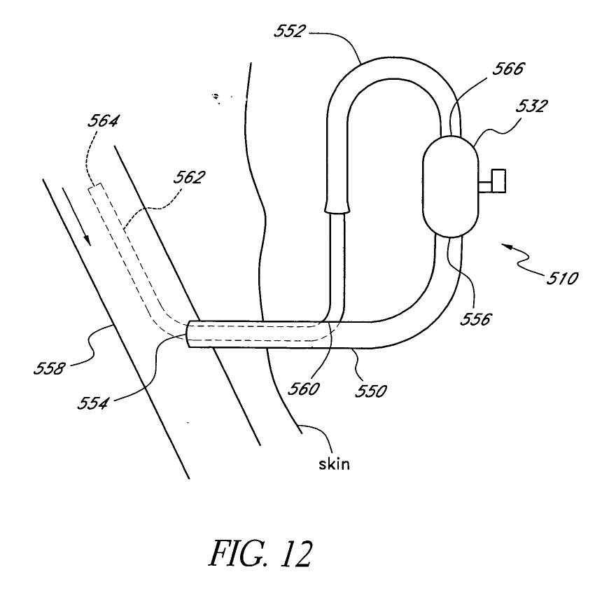

FIG. 12 shows another heart assist system 510 that takes further advantage of supplemental blood perfusion and heart load reduction benefits while remaining minimally invasive in application. The heart assist system 510 is an extracardiac pumping system that includes a pump 532, an inflow conduit 550 and an outflow conduit 552. In the illustrated embodiment, the inflow conduit 550 comprises a vascular graft. The vascular graft conduit 550 and the outflow conduit 552 are fluidly coupled to a pump 532. The pump 532 is configured to pump blood through the patient at subcardiac volumetric rates, and has an average flow rate that, during normal operation thereof, is substantially below that of the patient's heart when healthy. In one variation, the pump 532 may be a rotary pump. Other pumps described herein, or any other suitable pump can also be used in the extracardiac pumping system 510. In one application, the pump 532 is configured so as to be implantable.

The vascular graft 550 has a first end 554 and a second end 556. The first end 554 is sized and configured to couple to a non-primary blood vessel 558 subcutaneously to permit application of the extracardiac pumping system 510 in a minimally-invasive procedure. In one application, the vascular graft conduit 550 is configured to couple to the blood vessel 558 via an anastomosis connection.

The second end 556 of the vascular graft 550 is fluidly coupled to the pump 532 to conduct blood between the non-primary blood vessel 558 and the pump 532. In the embodiment shown, the second end 556 is directly connected to the pump 532, but, as discussed above in connection with other embodiments, intervening fluid conducting elements may be interposed between the second end 556 of the vascular graft 550 and the pump 532. Examples of arrangements of vascular graft conduits may be found in U.S. application Ser. No. 09/780,083, filed Feb. 9, 2001, entitled EXTRA-CORPOREAL VASCULAR CONDUIT, which is hereby incorporated by reference in its entirety and made a part of this specification.

FIG. 12 illustrates that the present inventive embodiment further comprises means for coupling the outflow conduit 552 to the vascular graft 550, which may comprise in one embodiment an insertion site 560. In the illustrated embodiment, the insertion site 560 is located between the first end 554 and the second end 556 of the vascular graft 550. The outflow conduit 552 preferably is coupled with a cannula 562. The cannula 562 can take any suitable form, e.g., defining a lumen with an inner size that increases distally.

In the illustrated embodiment, the insertion site 560 is configured to receive the cannula 562 therethrough in a sealable manner. In another embodiment, the insertion site 560 is configured to receive the outflow conduit 552 directly. The cannula 562 includes a first end 564 sized and configured to be inserted through the insertion site 560, through the cannula 550, and through the non-primary blood vessel 558. The conduit 552 has a second end 566 fluidly coupled to the pump 532 to conduct blood between the pump 532 and the blood vessel 558.

The extracardiac pumping system 510 can be applied to a patient, as shown in FIG. 12, so that the outflow conduit 552 provides fluid communication between the pump 532 and a location upstream or downstream of the location where the cannula 562 enters the non-primary blood vessel 558. In another application, the cannula 562 is directed through the blood vessel to a different blood vessel, upstream or downstream thereof. Although the vascular graft 550 is described above as an “inflow conduit” and the conduit 552 is described above as an “outflow conduit,” in another application of this embodiment, the blood flow through the pumping system 510 is reversed (i.e., the pump 532 pumps blood in the opposite direction), whereby the vascular graft 550 is an outflow conduit and the conduit 552 is an inflow conduit.

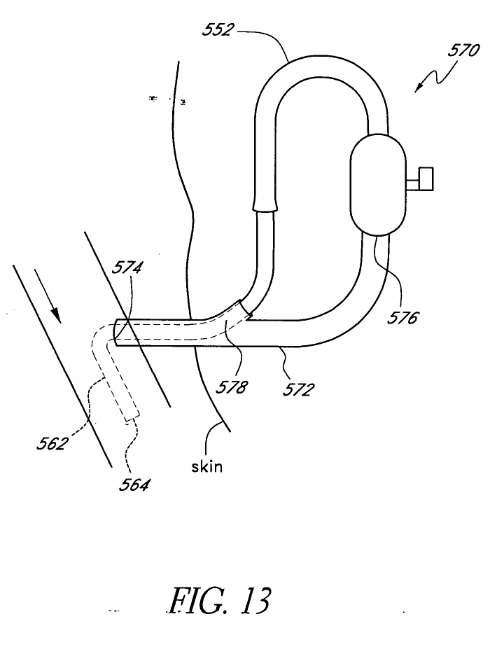

FIG. 13 shows a variation of the extracardiac pumping system shown in FIG. 12. In particular, a heart assist system 570 includes an inflow conduit 572 that comprises a first end 574, a second end 576, and means for connecting the outflow conduit 552 to the inflow conduit 572. In one embodiment, the inflow conduit 572 comprises a vascular graft. The extracardiac pumping system 570 is otherwise similar to the extracardiac pumping system 510. The means for connecting the conduit 552 to the inflow conduit 572 may comprise a branched portion 578. In one embodiment, the branched portion 578 is located between the first end 574 and the second end 576. The branched portion 578 is configured to sealably receive the distal end 564 of the outflow conduit 552. Where, as shown, the first end 564 of the outflow conduit 552 comprises the cannula 562, the branched portion 578 is configured to receive the cannula 562. The inflow conduit 572 of this arrangement comprises in part a multilumen cannula, where the internal lumen extends into the blood vessel 558. Other multilumen catheter arrangements are shown in U.S. application Ser. No. 10/078,283, incorporated by reference herein above.

2. Single-Site Application of Intravascular Pumping Systems

FIGS. 14-16 illustrate extracardiac heart assist systems that employ intravascular pumping systems. Such systems take further advantage of the supplemental blood perfusion and heart load reduction benefits discussed above while remaining minimally invasive in application. Specifically, it is contemplated to provide an extracardiac pumping system that comprises a pump that is sized and configured to be at least partially implanted intravascularly in any location desirable to achieve those benefits, while being insertable through a non-primary vessel.

FIG. 14 shows a heart assist system 612 that includes a pumping means 614 comprising preferably one or more rotatable impeller blades 616, although other types of pumping means 614 are contemplated, such as an Archimedes screw, a worm pump, or other means by which blood may be directed axially along the pumping means from a location upstream of an inlet to the pumping means to a location downstream of an outlet from the pumping means. Where one or more impeller blades 616 are used, such as in a rotary pump, such impeller blades 616 may be supported helically or otherwise on a shaft 618 within a housing 620. The housing 620 may be open, as shown, in which the walls of the housing 620 are open to blood flow therethrough. The housing 620 may be entirely closed, if desired, except for an inlet and outlet (not shown) to permit blood flow therethrough in a more channeled fashion. For example, the housing 620 could be coupled with or replaced by a cannula with a lumen that has an inner size that increases distally. The heart assist system 612 serves to supplement the kinetic energy of the blood flow through the blood vessel in which the pump is positioned, e.g., the aorta 16.

The impeller blade(s) 616 of the pumping means 614 of this embodiment may be driven in one of a number of ways known to persons of ordinary skill in the art. In the embodiment shown in FIG. 14, the impeller blade(s) 616 are driven mechanically via a rotatable cable or drive wire 622 by driving means 624, the latter of which may be positioned corporeally (intra- or extra-vascularly) or extracorporeally. As shown, the driving means 624 may comprise a motor 626 to which energy is supplied directly via an associated battery or an external power source, in a manner described in more detail herein. It is also contemplated that the impeller blade(s) 616 be driven electromagnetically through an internal or external electromagnetic drive. Preferably, a controller (not shown) is provided in association with this embodiment so that the pumping means 614 may be controlled to operate in a continuous and/or pulsatile fashion, as described herein.

Variations of the intravascular embodiment of FIG. 14 are shown in FIGS. 15 and 16. In the embodiment of FIG. 15, an intrasvascular extracardiac system 642 comprises a pumping means 644, which may be one of several means described herein. The pumping means 644 may be driven in any suitable manner, including means sized and configured to be implantable and, if desired, implantable intravascularly, e.g., as discussed above. For a blood vessel (e.g., descending aorta) having a diameter “A”, the pumping means 644 preferably has a significantly smaller diameter “B”. The pumping means 644 may comprise a pump 646 having an inlet 648 and an outlet 650. The pumping means 644 also comprises a pump driven mechanically by a suitable drive arrangement in one embodiment. Although the vertical arrows in FIG. 15 illustrate that the pumping means 644 pumps blood in the same direction as the flow of blood in the vessel, the pumping means 644 could be reversed to pump blood in a direction generally opposite of the flow in the vessel.

In one embodiment, the pumping means 644 also includes a conduit 652 in which the pump 646 is housed. The conduit 652 may be relatively short, as shown, or may extend well within the designated blood vessel or even into an adjoining or remote blood vessel at either the inlet end, the outlet end, or both. The intravascular extracardiac system 642 may further comprise an additional parallel-flow conduit, as discussed below in connection with the system of FIG. 16.

The intrasvascular extracardiac system 642 may further comprise inflow and/or outflow conduits or cannulae (not shown) fluidly connected to the pumping means 644, e.g., to the inlet and outlet of pump 646. Any suitable conduit or cannula can be employed. For example, a cannula defining a lumen with an inner size that increases distally could be coupled with an intrasvascular extracardiac system.

In another embodiment, an intrasvascular pumping means 644 may be positioned within one lumen of a multilumen catheter so that, for example, where the catheter is applied at the left femoral artery, a first lumen may extend into the aorta proximate the left subclavian and the pumping means may reside at any point within the first lumen, and the second lumen may extend much shorter just into the left femoral or left iliac. Such a system is described in greater detail in U.S. application Ser. No. 10/078,283, incorporated by reference herein above.

FIG. 16 shows a variation of the heart assist system of FIG. 15. In particular the intravascular system may further comprise an additional conduit 660 positioned preferably proximate the pumping means 644 to provide a defined flow path for blood flow axially parallel to the blood flowing through the pumping means 644. In the case of the pumping means 644 of FIG. 16, the means comprises a rotatable cable 662 having blood directing means 664 supported therein for directing blood axially along the cable. Other types of pumping means are also contemplated, if desired, for use with the additional conduit 660.

The intravascular extracardiac system described herein may be inserted into a patient's vasculature by any means known to those of ordinary skill, or by any obvious variant thereof. In one method of use, such a system is temporarily housed within a catheter that is inserted percutaneously, or by surgical cutdown, into a non-primary blood vessel and advanced through to a desired location. The catheter preferably is then withdrawn away from the system so as not to interfere with operation of the system, but to still permit the withdrawal of the system from the patient when desired. Further details of intravascular pumping systems may be found in U.S. patent application Ser. No. 10/686,040, filed Oct. 15, 2003, which is hereby incorporated by reference herein in its entirety.

C. Potential Enhancement of Systemic Arterial Blood Mixing

An advantage of the cardiac assist systems described above is the potential to enhance mixing of systemic arterial blood, particularly in the aorta. Such enhanced mixing ensures the delivery of blood with higher oxygen-carrying capacity to organs supplied by arterial side branches off of the aorta. A method of enhancing mixing using the cardiac assist systems described above preferably includes taking steps to assess certain parameters of the patient and then to determine the minimum output of the pump that, when combined with the heart output, ensures turbulent flow in the aorta, thereby enhancing blood mixing.

Blood flow in the aortic arch during normal cardiac output may be characterized as turbulent in the end systolic phase. Turbulence in a flow of fluid enhances the uniform distribution of particles within the fluid. It is believed that turbulence in the descending aorta enhances the homogeneity of blood cell distribution in the aorta. Laminar flow of viscous fluids leads to a higher concentration of particulate in the central portion of the flow. It is believed that, in low flow states such as that experienced during heart failure, there is reduced or inadequate mixing of blood cells leading to a lower concentration of nutrients at the branches of the aorta to peripheral organs and tissues. As a result, the blood flowing into branch arteries off of the aorta will likely have a lower hematocrit, especially that flowing into the renal arteries, the celiac trunk, the spinal arteries, and the superior and inferior mesenteric arteries, because these branches draw from the periphery of the aorta. The net effect of this phenomenon is that the blood flowing into these branch arteries has a lower oxygen-carrying capacity, because oxygen-carrying capacity is directly proportional to both hematocrit and the fractional 02 saturation of hemoglobin. Under those circumstances, these organs may experience ischemia-related pathology.

The phenomenon of blood streaming in the aorta, and the resultant inadequate mixing of blood resulting in central lumenal concentration of blood cells, is believed to occur when the Reynolds number (N_{R}) for the blood flow in the aorta is below 2300. To help ensure that adequate mixing of blood will occur in the aorta to prevent blood cells from concentrating in the center of the lumen, a method of applying the cardiac assist systems described above to a patient may also include steps to adjust the output of the pump to attain turbulent flow within the descending aorta upstream of the organ branches; i.e., flow exhibiting a peak Reynolds number of at least 2300 within a complete cycle of systole and diastole. Because flow through a patient is pulsatile in nature, and not continuous, consideration is preferably given to how frequently the blood flow through the aorta has reached a certain desired velocity and, thus, a desired Reynolds number. The method contemplated herein, therefore, may also include the step of calculating the average Womersley number (N_{W}), which is a function of the frequency of the patient's heart beat. Preferably, a peak Reynolds number of at least 2300 is attained when the corresponding Womersley number for the same blood flow is approximately 6 or above.

More specifically, the method may comprise calculating the Reynolds number for the blood flow in the descending aorta by determining the blood vessel diameter and both the velocity and viscosity of the fluid flowing through the aorta. The Reynolds number may be calculated pursuant to the following equation:

$${N}_{R} = \frac{V \cdot d}{υ}$$

where: V=the velocity of the fluid; d=the diameter of the vessel; and ν=the viscosity of the fluid. The velocity of the blood flowing through the aorta is a function of the cross-sectional area of the aorta and the volume of flow therethrough, the latter of which is determined both by the patient's own cardiac output and by the output of the extra cardiac pump. Velocity may be calculated by the following equation:

$$V = \frac{Q}{π {r}^{2}}$$

where Q=the volume of blood flowing through the blood vessel, e.g., the aorta, per unit time; and r=the radius of the vessel. If the relationship between the pump output and the velocity is already known or independently determinable, the volume of blood flow Q may consist only of the patient's cardiac output, with the knowledge that that output will be supplemented by the subcardiac pump. If desired, however, the cardiac assist system can be implemented and applied to the patient first, before calculating Q, which would consist of the combination of cardiac output and the pump output.

The Womersley number may be calculated as follows:

$${N}_{W} = r \sqrt{\frac{2 πω}{υ}}$$

where r is the radius of the vessel being assessed, ω is the frequency of the patient's heartbeat, and ν=the viscosity of the fluid. For a peak Reynolds number of at least 2300, a Womersley number of at least 6 is preferred, although lower values, such as 5, would also be acceptable.

By determining (i) the viscosity of the patient's blood, which is normally about 3.0 mm²/sec (kinematic viscosity), (ii) the cardiac output of the patient, which of course varies depending upon the level of CHF and activity, and (iii) the diameter of the patient's descending aorta, which varies from patient to patient but is about 21 mm for an average adult, one can determine the flow rate Q that would result in a velocity through the aorta necessary to attain a Reynolds number of at least 2300 at its peak during the patient's heart cycle. Based upon that determination of Q, one may adjust the output of the pump to attain the desired turbulent flow characteristic through the aorta, enhancing mixing of the blood therethrough.

One may use ultrasound (e.g., echocardiography or abdominal ultrasound) to measure the diameter of the aorta, which is relatively uniform in diameter from its root to the abdominal portion of the descending aorta. One may measure cardiac output using a thermodilution catheter or other techniques known to those of skill in the art. Finally, one may measure viscosity of the patient's blood by using known methods; for example, using a capillary viscosimeter. In many cases, the above methods will provide a basis to more finely tune the system to more optimally operate the system to the patient's benefit. Other methods may include steps to assess other patient parameters that enable a person of ordinary skill in the art to optimize the cardiac assist system to ensure adequate mixing within the vascular system of the patient.

Alternative methods that provide the benefits discussed herein include the steps of, prior to applying a shape change therapy, applying a blood supplementation system (such as one of the many examples described herein) to a patient, whereby the methods are designed to improve the ability to reduce the size and/or wall stress of the left ventricle, or both ventricles, thus reducing ventricular loading. Specifically, one example of such a method comprises the steps of providing a pump configured to pump blood at subcardiac rates, providing inflow and outflow conduits configured to fluidly communicate with non-primary blood vessels, fluidly coupling the inflow conduit to a non-primary blood vessel, fluidly coupling the outflow conduit to the same or different (primary or non-primary) blood vessel and operating the subcardiac pump in a manner, as described herein, to reduce the load on the heart, wherein the fluidly coupling steps may comprise anastomosis, percutaneous cannulazation, positioning the distal end of one or both conduits within the desired terminal blood vessel or any combination thereof. The method further comprises, after sufficient reduction in ventricular loading, applying a shape change therapy in the form of, for example, a cardiac reshaping device, such as those referred to herein, or others serving the same or similar function, for the purpose of further reducing the size of and/or wall stress on one or more ventricles and, thus, the heart, and/or for the purpose of maintaining the patient's heart at a size sufficient to enhance recovery of the patient's heart.

D. Apparatus and Methods For Reducing Bleeding From A Cannulation Site

As discussed above, the inflow and outflow conduits of an extracardiac heart assist system may be connected to one or more cannula(e) that are in fluid communication with the patient's vasculature. For example, FIG. 1 shows that the inflow conduit 50 has an end 58 that is coupled with a first non-primary blood vessel (e.g., the left femoral artery 26) by way of an inflow cannula 60. Depending upon how it is applied, the cannulation may generate sufficient bleeding to form a hematoma. The risk of hematoma formation is greater when the cannula penetrates an artery, because of the higher pressure in arteries. A hematoma is not only unsightly, but it can also be quite painful for the patient. The bleeding may be exacerbated by an anticoagulation protocol established as part of the cardiac assistance therapy. Further, the longer a cannula is resident in the patient, the greater the risk that a hematoma will form.

Referring to FIGS. 17, 18 and 23, the present invention further comprises apparatus for reducing bleeding at a cannulation site. The inventive apparatus comprises a cannula shaft 700 having a hub 702 attached to a proximal end thereof. A plug 704 is disposed about the cannula 700 just distal of the hub 702. The plug 704 of the apparatus of FIG. 17 is preferably shaped as a tube having an interior lumen (not shown) that snugly receives the cannula shaft 700. The plug 704 reduces bleeding from the cannulation site, as described in detail below.

In one embodiment, an exterior surface 710 of the plug 704 defines a generally circular cross-section. However, those of skill in the art should appreciate that the plug exterior surface 710 could define other shapes in cross-section, such as a hexagon or an octagon. The diameter of the plug outer surface 710 may be uniform or tapered; for example, tapered from a proximal end 713 of the plug to a distal end 715. In order to minimize trauma to the tissue 714 between the skin 716 and the vessel wall 718, the plug outer size is preferably no greater than necessary to achieve the advantages described below. In FIG. 17, the difference in exterior dimensions between the cannula 700 and the plug 704 has been exaggerated for illustrative clarity.

In one embodiment, a distal face 720 of the plug 704 preferably defines an exterior dimension that is greater than the outer diameter of the cannula 700 by at least a certain threshold value. The plug distal face 720 thus defines a shoulder with respect to the cannula outer surface 724. In one embodiment, the outer dimension of the plug 704 at the distal end 715 is at least 0.1 mm greater than the outer diameter of the cannula 700. However, those of skill in the art should appreciate that the plug distal end outer dimension could be closer to the cannula outer diameter.

Still referring to FIG. 17, the cannula 700 may be used in a manner to penetrate the vessel wall 718 such that the cannula shaft 700 is disposed within the vessel. The cannula hub 702 preferably remains outside the patient. In use, the plug 704 may penetrate the patient's skin 716 and the tissue 714 that lies between the skin 716 and the vessel wall 718. However, the plug 704 does not penetrate the vessel wall 718. The exterior dimension of the plug distal face 720 prevents the plug 704 from advancing through the vessel wall 718.

In the position shown in FIG. 17, the distal face 720 of the plug 704 is configured so as to be positioned adjacent to the outer surface of the vessel wall 718. In some embodiments, the distal face 720 is configured to conform to the vessel wall 718. In one technique, the cannula 700 is applied such that the distal face 720 does not impinge upon the vessel wall 718 in a manner that substantially impairs blood flow through the vessel to the vasculature downstream of the penetration site. In one technique, the vessel is considered not to be substantially impaired as long as the patient does not experience ischemia in downstream tissue. In some techniques, the vessel is considered not to be substantially impaired by the application of the cannula 700 if eighty-five percent or more of the flow in the vasculature downstream of the penetration site is maintained compared to application of a cannula without the plug 704. In some techniques, the vessel is considered not to be substantially impaired by the application of the cannula 700 if ninety percent or more of the flow in the vasculature downstream of the penetration site is maintained compared to application of a cannula without the plug 704. In some techniques, the vessel is considered not to be substantially impaired by the application of the cannula 700 if ninety-five percent or more of the flow in the vasculature downstream of the penetration site is maintained compared to application of a cannula without the plug 704. One technique for minimizing the impairment of flow to the downstream vasculature involves applying the cannula 700 such that the distal face 720 applies little or no pressure to the vessel wall 718.

In some embodiments, the plug 704 is configured and applied such that it does not apply an axial force to tissues between the vessel wall 718 and the skin. In some embodiments, the plug 704 is applied so that a radial force is applied by the plug 704 to tissue between the vessel wall 718 and the skin. For example, the plug 704 can be configured to expand to a size larger than a dilator inserted through the skin to put a radial force or pressure on the tissue. When a radial force or pressure is applied by the plug 704 against the tissue, the pressure reduces bleeding, e.g., in the tissues between the vessel and the skin. FIG. 17 shows that the hub 702 preferably is positioned above the skin 716, e.g., outside the body. In some techniques, the hub 702 is taped or sutured onto the patient. Over time, blood clots around the cannula 700 and plug 704. The clotted blood holds the cannula 700 and plug 704 in place, alleviating the need for the suture.

An inventive method of reducing bleeding by using the apparatus of FIG. 17 is contemplated and explained below. Those of skill in the art should appreciate that other methods of using the inventive apparatus could be practiced instead. Those of skill in the art should further appreciate that the manner of insertion described herein is not intended to limit the scope of the claims below.

One method contemplated herein comprises the step of advancing an introducer needle (not shown) through a patient's skin 716 and tissue 714 to pierce the vessel wall 718 and create a vessel opening (not shown). The method further comprises the steps of advancing a guide wire (not shown) through the needle bore until a distal end of the guide wire is disposed within the target vessel, and then removing the needle.

The method further comprises the step of dilating the tissue 714 and the vessel opening by advancing at least one dilator (not shown) over the guide wire. More than one dilator may be used in this step, with each successive dilator being slightly larger (a process known as step dilation). Once the tissue 714 and vessel opening have been sufficiently dilated, the next step comprises advancing the cannula 700 over the guide wire until a distal end 726 of the cannula 700 is disposed within the vessel.

The cannula 700 and the plug 704 may be advanced at the same time. Further advancement may be halted when the plug 704 reaches the configuration shown in FIG. 17. Alternatively, before the cannula 700 is advanced, the plug 704 may first be advanced into the patient's tissue until the distal face 720 of the plug 704 abuts the vessel wall 718. The cannula 700 is then advanced through the plug lumen and into the vessel.

When cannulating a vessel, the cannula generally penetrates the patient's skin at an obtuse angle. Accordingly, in one embodiment of the present apparatus, the distal face 720 of the plug 704 defines a plane with which a longitudinal axis of the cannula 700 forms a non-perpendicular angle. The plug 704 is thus able to flushly abut the vessel wall 718. Sometimes, however, the cannula 700 and plug 704 may not be oriented at just the right angle to achieve the desired flushness between the plug distal face 720 and the vessel wall 718. Thus, in one embodiment of the present apparatus, at least the plug distal end 715 is constructed of a pliant material. In this embodiment the pliant distal end 715 of the plug 704 is able to conform to the vessel wall 718 and achieve a flush engagement regardless of whether the plug 704 is oriented at just the right angle. Preferred materials for the plug distal end 715 include neoprene, polyvinylchloride (PVC) having a relatively low durometer, and silicone.

As a cannula is advanced through tissue, it may encounter significant resistance. Under those circumstances, the plug 704 is also likely to encounter significant resistance as it advances through the patient's tissue 714. To allow the plug 704 to advance, in one embodiment at least the proximal portion 713 thereof is constructed of a relatively rigid material. Preferred materials for the plug proximal portion 713 include polyethylene, PVC having a relatively high durometer, and PTFE.

In one alternative embodiment, the plug 704 may be initially in a compressed configuration prior to being inserted into the patient. At a certain point after insertion, the plug 704 expands to the desired exterior dimensions. For example, the plug 704 could be constructed of a compressible material that is compressed and then coated with a biocompatible material that dissolves upon exposure to certain bodily fluids, such as blood. Upon insertion, the bodily fluid(s) dissolve the coating, allowing the plug 704 to expand. The coating could be configured to dissolve fully only after a certain amount of time has passed, thereby preventing premature expansion of the plug 704. The coating can be of any suitable material, such as any of a variety of hydrophilic materials, e.g., polyurethane or a hydrogel.

In order to further reduce bleeding from the vessel opening and/or the tissue 714 between the patient's skin 716 and the vessel wall 718, the plug 704 may include a hemostatic agent. For example, if the plug distal face 720 is coated with a hemostatic agent, the agent promotes clotting of any blood coming into contact with it. Additional portions of the plug 704 may also be coated with a hemostatic agent. For example, the entire outer surface of the plug 704 may be coated with a hemostatic agent. Examples of possible hemostatic agents include fibrin glue, for example, TISSEAL™ (GENERIC TERM) sold by Baxter. However, those of skill in the art should appreciate that a variety of other hemostatic agents could be used instead.

FIG. 23 illustrates another embodiment of the present apparatus for reducing bleeding at a cannulation site. The apparatus of FIG. 23 is substantially identical to the apparatus of FIG. 17. However, the plug 703 includes a second shoulder 705 located proximally of the first shoulder 720, where the second shoulder 705 is defined by an increase in the exterior dimension of the plug 703. Alternatively, the second shoulder 705 may comprise a separate component that is disposed about a proximal end of the plug 703.

The portion of the plug 703 that is proximal the second shoulder 705 is adapted to remain outside the patient's skin 716, as illustrated in FIG. 23. Just as the first shoulder 720 may apply pressure to the patient's vessel 718, preferably without substantially impairing blood flow downstream, the second shoulder 705 is configured so as to apply pressure to the patient's skin 716. The pressure may reduce bleeding from the cannulation site. The second shoulder 705 may include a hemostatic agent. The cannula 700 and the variations thereof described herein can be modified to incorporate one or more features or incorporated into systems by being combined with one or more structures described in U.S. application Ser. No. 10/866,649, filed Jun. 10, 2004, in U.S. application Ser. No. 10/866,535, filed Jun. 10, 2004 or in U.S. application Ser. No. 10/865,045, filed Jun. 10, 2004, each of which is hereby expressly incorporated by reference in its entirety and made a part of this specification

FIG. 18 illustrates another embodiment of the present apparatus for reducing bleeding at a cannulation site. The apparatus of FIG. 18 shares many similarities with the apparatus of FIG. 17. However, the following discussion focuses on the differences between these two apparatus.

The apparatus of FIG. 18 comprises a standard cannula shaft 700a having a hub 702a attached to a proximal end thereof. A plug 706 is disposed about the cannula 700a just distal of the hub 702a. The plug 706 is preferably shaped as a tube having an interior lumen that is wider than a diameter of an outer wall of the cannula 700a. The cannula outer wall and plug inner wall thus define an annular passage 708, as further illustrated in FIG. 19.

The annular passage 708 is adapted to receive a hemostatic agent. A proximal end 713a of the plug 706 includes a port 728 through which a flowable hemostatic agent may be injected. In the illustrated embodiment, the port 728 comprises a luer connector. A syringe may engage the luer connector so that hemostatic agent may be injected into the annular passage 708. Those of skill in the art should appreciate that the port 728 could embody a number of other configurations.

As shown in FIG. 18, a hemostatic agent 730 injected through the port 728 flows along the length of the annular passage 708 (see arrows in FIG. 18) and exits the distal end 715 of the plug 706. The inventive apparatus is preferably configured so that the hemostatic agent 730 spreads out over the vessel wall 718 and promotes blood clotting in that region. To further promote blood clotting in the tissue 714 between the skin 716 and the vessel wall 718, the hemostatic agent 730 may be injected through the port 728 as the plug 706 is withdrawn from the cannulation site without dislodging the cannula 700a from the vasculature. In one approach, the cannula 700a can have additional length (e.g., between the skin and the hub 702a when applied), which can permit the plug 706 to translate proximally with little or no proximal movement of the cannula 700a. In another technique, the hemostatic agent 730 could be deployed while the cannula 700a and plug 706 are being inserted. In another technique, the hemostatic agent 730 could be deployed while the cannula 700a and plug 706 are being inserted and as the plug 706 is withdrawn, as discussed above. The injected hemostatic agent 730 continues to exit the plug distal end 715a and coats the tissue 714 between the skin 716 and the vessel wall 718. Once a sufficient coating has been applied, the plug 706 may be reinserted into the tissue 714 until it reaches the configuration of FIG. 18.

A method of using the apparatus of FIG. 18 is explained below. Those of skill in the art should appreciate that other methods of using the inventive apparatus could be practiced instead. Those of skill in the art will further appreciate that the manner of insertion described herein is not intended to limit the scope of the claims below.

One inventive method contemplated herein comprises the step of advancing an introducer needle (not shown) through a patient's skin 716 and tissue 714 to pierce the vessel wall 718 and create a vessel opening (not shown). The method further comprises the steps of advancing a guide wire (not shown) through the needle bore until a distal end of the guide wire is disposed within the vessel, and then removing the needle.

The next step comprises dilating the tissue 714 and the vessel opening by advancing at least one dilator (not shown) over the guide wire. More than one dilator may be used in this step, with each successive dilator being slightly larger (a process known as step dilation). Once the tissue 714 and vessel opening have been sufficiently dilated, the next step comprises advancing the cannula 700a over the guide wire until a distal end 726 of the cannula 700a is disposed within the vessel.

The cannula 700a and the plug 706 may be advanced at the same time. Further advance may be halted when the plug 706 reaches the configuration shown in FIG. 18. Alternatively, before the cannula 700a is advanced, the plug 706 may first be advanced into the patient's tissue until the distal end 715a of the plug 706 abuts the vessel wall 718. The cannula 700a is then advanced through the plug lumen and into the vessel.

The next step may comprise injecting a hemostatic agent 730 through the port 728. The hemostatic agent 730 flows according to the description above and promotes blood clotting near the vessel 718. To further promote blood clotting in the tissue 714 between the skin 716 and the vessel wall 718, the method may further comprise the step of injecting hemostatic agent 730 through the port 728 as the plug 706 is withdrawn from the cannulation site. The injected hemostatic agent 730 flows according to the description above and promotes blood clotting in the tissue 714 between the skin 716 and the vessel wall 718. Once a sufficient coating has been applied, the method may further comprise the step of reinserting the plug 706 into the tissue 714 until it reaches the configuration of FIG. 18.

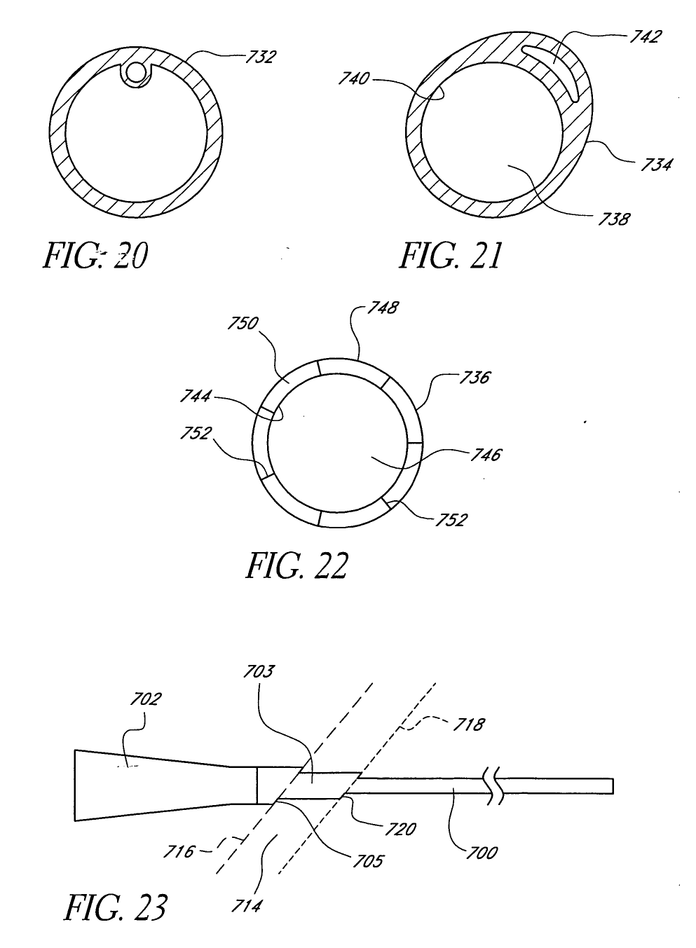

FIGS. 20-22 illustrate cross-sections of alternative plugs 732, 734, 736. As discussed below, the plugs can be coupled with a cannula in any suitable manner. For example, as discussed further below, the plugs 732, 734 each have a first lumen to receive a cannula and a second, separate lumen adapted to receive a hemostatic agent. In one arrangement, a cannula that has an outer periphery that corresponds to the inner periphery of the plug is received in the main lumen of the plugs 732, 734 (e.g., the larger lumen). An outer periphery of a cannula can correspond with an inner periphery of a plug by having the same shape in cross-section as the inner periphery, by being configured such that the plug and cannula are capable of relative movement, by being configured such that relative movement is prevented between the cannula and plug, or by being smaller than the plug inner periphery so that fluid may flow between the cannlua and plug, or in any other suitable manner. In one embodiment, there is not a separate structure defining the main, blood-flow lumen; the inside surface of the plug defines a portion of the blood flow lumen of the cannula.

The plug 734 of FIG. 21 includes a first cylindrical lumen 738 that is adapted to receive the cannula 700. This lumen 738 may be sized to snugly receive the cannula 700, or an inner wall 740 of the plug 734 may form an annular passageway together with an outer wall of the cannula 700, as in the embodiment of FIG. 19. The plug 734 of FIG. 21 includes a second lumen 742 that is radially offset from the first lumen 738. The cross-section of the second lumen 742 may be substantially flat, as shown, or may embody virtually any other shape. The second lumen 742 is adapted to receive the flowable hemostatic agent 730 in the same manner described above.

The plug 736 of FIG. 22 includes an inner wall 744 defining a first cylindrical lumen 746. The first lumen 746 is adapted to receive the cannula 700. This lumen 746 may be sized to snugly receive the cannula 700, or the inner wall 744 of the plug 736 may form an annular passageway together with an outer wall of the cannula 700, as in the embodiment of FIG. 19. The plug 736 of FIG. 22 includes an outer wall 748 that forms an annular passageway 750 together with the inner wall 744.

A plurality of dividers 752 extend between the inner wall 744 and the outer wall 748. The dividers 752 subdivide the annular passageway 750 into a plurality of sections, with each section having an arc-shaped cross-section. In the illustrated embodiment, seven dividers 752 are provided. However, those of skill in the art will appreciate that fewer or more dividers 752 could be provided.

The subdivided annular passageway 750 is adapted to receive the flowable hemostatic agent 730 in the same manner described above. The dividers may help to evenly distribute the hemostatic agent 730 so that when the hemostatic agent 730 exits the plug distal end 715, it spreads out uniformly around the vessel opening, and onto all areas of the tissue 714 between the skin 716 and the vessel wall 718.

### Scope of the Invention

The above presents a description of the best mode contemplated for carrying out the present apparatus and methods for reducing bleeding from a cannulation site, and of the manner and process of making and using the same, in such full, clear, concise, and exact terms as to enable any person skilled in the art to which they pertain to make and use these apparatus and methods. These apparatus and methods are, however, susceptible to modifications and alternate constructions from that discussed above that are fully equivalent. Consequently, these apparatus and methods are not limited to the particular embodiments disclosed. On the contrary, these apparatus and methods cover all modifications and alternate constructions coming within the spirit and scope of the apparatus and methods as generally expressed by the following claims, which particularly point out and distinctly claim the subject matter of the apparatus and methods.

What is claimed is:

1. An apparatus to reduce or prevent bleeding from a percutaneous cannula insertion site, thereby reducing a risk of hematoma formation, the apparatus comprising:
a cannula having a proximal end and a distal end, the cannula being configured to extend into a blood vessel and provide a path to an interior of the vessel; and
a plug disposed about and at least partially overlapping a portion of the cannula, the plug having a distal end configured to abut an exterior wall of the vessel while the cannula is maintained within the vessel and to reduce or prevent bleeding from the vessel.

2. The apparatus of claim 1, wherein the plug distal end defines a shoulder relative to the cannula.

3. The apparatus of claim 1, further comprising a hemostatic agent applied to the plug distal end, the hemostatic agent configured to further reduce bleeding from the vessel.

4. The apparatus of claim 1, further comprising a hub disposed about a proximal end of the cannula, such that the plug is located distally of the hub.

5. The apparatus of claim 1, wherein the plug is generally tubular.

6. The apparatus of claim 1, wherein at least the plug distal end is constructed of a relatively compliant material.

7. The apparatus of claim 6, wherein a proximal portion of the plug is constructed of a relatively rigid material.

8. The apparatus of claim 1, wherein the plug distal end defines a plane that is not perpendicular to a longitudinal axis of the plug.

9. The apparatus of claim 1, wherein the plug further comprises a first portion having a first exterior dimension, and a second portion having a second, larger, exterior dimension, a distal face of the second portion forming a shoulder with respect to the first portion.

10. The apparatus of claim 9, wherein the shoulder is configured to abut skin in the vicinity of the percutaneous cannula insertion site and apply pressure thereto while the cannula is maintained within the vessel, to thereby reduce or prevent bleeding from the site.

11. An apparatus to reduce or prevent bleeding from a percutaneous cannula insertion site, thereby reducing a risk of hematoma formation, the apparatus comprising:
a cannula configured to extend into a blood vessel and provide a path through a vessel opening to an interior of the vessel;
a plug configured to accept a flow of hemostatic agent therethrough, the plug being disposed about and at least partially overlapping a portion of the cannula, an exterior wall of the cannula and an interior wall of the plug defining an annular space.

12. The apparatus of claim 11, further comprising a hemostatic agent that is configured to flow through the annular space and out of a distal end of the plug.

13. The apparatus of claim 12, wherein the hemostatic agent is configured to promote hemostasis at the vessel opening and/or within tissue surrounding the opening.

14. The apparatus of claim 11, wherein a proximal end of the plug includes an access port.

15. The apparatus of claim 14, wherein the access port comprises a luer connector.

16. The apparatus of claim 11, further comprising a hub disposed about a proximal end of the cannula, such that the plug is located distally of the hub.

17. The apparatus of claim 11, wherein side walls of the plug include at least one aperture.

18. The apparatus of claim 17, wherein the plug side walls include a plurality of apertures.

19. The apparatus of claim 17, further comprising a hemostatic agent that is configured to flow through the annular space and out of the apertures, wherein upon exiting the plug, the hemostatic agent promotes hemostasis at the vessel opening and/or within tissue surrounding the opening.

20. The apparatus of claim 19, wherein a proximal end of the plug includes an access port.

21. A method of reducing or eliminating bleeding from a percutaneous cannula insertion site, thereby reducing a risk of hematoma formation, the method comprising the steps of:
inserting a cannula, having a plug disposed about a portion thereof, into a vessel such that the cannula provides a path through a vessel opening to an interior of the vessel; and
advancing the cannula into the vessel until a distal end of the plug abuts an outer wall of the vessel surrounding the vessel opening.

22. The method of claim 21, wherein a size of the vessel opening is substantially equal to an outer dimension of the cannula, such that the cannula can penetrate the opening but the plug is substantially prevented from penetrating the opening.

23. The method of claim 22, further comprising the step of applying a force along a longitudinal axis of the cannula, such that the plug distal end applies pressure to the vessel outer wall without substantially impairing blood flow downstream of the penetration site.

24. The method of claim 21, wherein the plug distal end includes a hemostatic agent.

25. The method of claim 21, wherein the plug distal end lies in a plane that is not perpendicular to a longitudinal axis of the cannula.

26. A method of reducing or eliminating bleeding from a percutaneous cannula insertion site, thereby reducing a risk of hematoma formation, the method comprising the steps of:
inserting a cannula, having a plug disposed about a portion thereof, an outer surface of the cannula and an inner surface of the plug defining boundaries of an annular space, into a vessel such that the cannula provides a path through a vessel opening to an interior of the vessel
advancing the cannula into the vessel until a distal end of the plug abuts an outer wall of the vessel surrounding the vessel opening; and
injecting a hemostatic agent into the annular space.

27. The method of claim 26, further comprising the step of forcing the hemostatic agent through the annular space such that the hemostatic agent exits the annular space through the plug distal end.

28. The method of claim 27, wherein the plug further includes at least one aperture in a side wall thereof.

29. The method of claim 28, wherein at least a portion of the hemostatic agent exits the annular space through the at least one aperture.

30. The method of claim 26, wherein a proximal end of the plug includes an input port through which the hemostatic agent may be introduced into the annular space.

31. The method of claim 30, wherein the input port comprises a luer connector.

32. The method of claim 26, further comprising the step of withdrawing the plug from the percutaneous cannula insertion site while maintaining the cannula within the vessel opening.

33. The method of claim 32, further comprising the step of forcing the hemostatic agent through the annular space, such that as the plug is withdrawn from the percutaneous cannula insertion site, the hemostatic agent flows through the distal end of the plug to promote hemostasis in tissue surrounding the vessel opening.

34. A medical apparatus comprising:
a cannula configured to extend into a blood vessel through a percutaneous insertion site and provide a path to an interior of the vessel; and
means for reducing bleeding from the insertion site.
